(19) United States
(12) Patent Application Publication
(10) Pub. No.: US 2014/0193900 A1
(43) Pub. Date: Jul. 10, 2014

(54) DEVICE FOR WASHING SUSPENDED CELLS OR PARTICLES

(71) Applicants: Ivar Meyvantsson, Madison, WI (US); Steve Hayes, Madison, WI (US); Victoria Echeverria, Madison, WI (US); Robert Lowery, Madison, WI (US); Casey Lamers, Madison, WI (US)

(72) Inventors: Ivar Meyvantsson, Madison, WI (US); Steve Hayes, Madison, WI (US); Victoria Echeverria, Madison, WI (US); Robert Lowery, Madison, WI (US); Casey Lamers, Madison, WI (US)

(21) Appl. No.: 14/124,900

(22) PCT Filed: May 29, 2012
(86) PCT No.: PCT/US12/39873

§ 371 Date: Mar. 10, 2014

Related U.S. Application Data
(60) Provisional application No. 61/495,239, filed on Jun. 9, 2011.

(51) Int. Cl. C12M 1/00; G01N 1/34; C12N 5/00

(52) U.S. Cl. CPC C12M 47/12; C12N 5/0068; G01N 1/34
USPC 435/325; 435/308.1; 435/7.1

(57) ABSTRACT

A device and method are disclosed to enable fluid manipulations without disturbing or removing particles or cells. By engineering assay compartment geometries to take advantage of fluid mechanical effects, the flow velocity may be diminished around particles or cells. This technology may be used to process particles or cells of various kinds that are suspended in liquids or gels, and is suitable for use in high throughput screening.

### CROSS-REFERENCE TO RELATED APPLICATIONS

This application claims priority from U.S. provisional patent application Ser. No. 61/495,239 filed Jun. 9, 2011. This application is incorporated herein by reference in its entirety.

### STATEMENT REGARDING FEDERALLY SPONSORED RESEARCH OR DEVELOPMENT

This invention was made with United States government support under grant number R44 HL088785 awarded by the following government agency: National Heart, Lung and Blood Institute. The United States government has certain rights in this invention.

### FIELD OF THE INVENTION

This application relates to devices and methods for cell and particle washing. In particular, this application discloses devices and methods for restricting fluid velocity and the accompanying forces acting on particles or cells by the inclusion of structural components within a tissue culture well or dish.

### BACKGROUND OF THE INVENTION

A variety of tubes, flasks, dishes, and assay plates are used as containers for cells during cell biology experimentation. While many cells adhere to the surface of these vessels, others are naturally non-adherent (e.g., bone marrow, peripheral blood, apoptotic cells, microorganisms) or are most easily or appropriately analyzed in suspension (e.g., virus-infected airway cells). There is often a need to apply reagents to these cells and then wash these reagents away later, such as for controlled exposure to dissolved compounds or biomolecules, transfection, pulse-chase experiments, labeling, fixing, and staining. Washing cells historically has involved placing the cell suspension into a tube or plate and pelleting the cells to the bottom through the use of a centrifuge. Then, a supernatant is decanted or pipetted from the tube or well, a wash liquid is added, the cell pellet is resuspended and these steps are repeated multiple times.

However, such conventional cell washing techniques are not readily adapted to a high through screening (HTS) scale which allow for automated, high volume processing of test samples. Because such systems utilize arrays of wells, periodic removal and replacement of culture, washing, labeling, or staining solutions or other manipulation of the fluid environment in at HTS scale is difficult without, at the same time, disturbing and/or removing non-adherent cells which are suspended in the fluid.

Hence, a need exists for a well-based systems that is compatible with standard robotic cell and assay systems for conveniently washing individual cell populations in a high throughput.

### SUMMARY OF THE INVENTION

The instant invention provides an improved device and method to exchange the liquid solution around particles or cells, commonly referred to as washing. The particles or cells are protected by geometric constructs that limit fluid convection in the vicinity of the particles or cells while, at the same time, allowing efficient replacement of the bulk fluid in the assay wells. Thus, the washing of particles or cells in aqueous suspension, and in particular non-adherent cells or particles, may be carried out in such a way as to protect the particles or cells from being disturbed by washing.

In one form, particles or cells are delivered to the device in liquid suspension and allowed to settle to the lowest surface of a patterned structure (e.g., a flat area in-between arrayed posts) at the bottom of the well. After the desired incubation time with the reagent of interest, the liquid above the posts is removed, and replaced with the washing liquid. The volume of liquid remaining among the posts after the removal and before the addition is much smaller than that removed and added. Due to the relative sizes of this small volume and the volume above the posts, the two quickly equilibrate via diffusion.

The fluid mechanical effects at play can be described as follows. When a liquid flows across a solid surface, there will be a shear force at the boundary between the liquid and solid. This shear force imposes a drag on the flow and slows it down in the vicinity of the surface. Structures protruding from the surface will extend the range of this effect to a greater distance above the surface. Given appropriate feature design and operational parameters, the flow velocity at the surface can be made essentially zero, while the flow velocity in the fluid above the features can be large enough to efficiently add and remove a significant volume within a reasonable period of time.

As a non-limiting example of patterned structures, consider an array of posts with a certain height, diameter, spacing, in a close-packed arrangement (i.e. with six nearest neighbors at 60° angles), and protruding from a flat, solid surface. Such a patterned structure may be prepared such that particles or cells of a given diameter can settle in or rest between the posts. Given a protocol with the appropriate combination of liquid viscosity, liquid density, post geometry, particle size, and flow velocity, the liquid above the posts can be removed and replaced with negligible disturbance to the particles.

As a further non-limiting example, consider locating these features at the bottom of each well in a multi-well or microtiter plate. In this case, the fluid flow may be actuated by dispensing and aspirating from a pipette or an automated liquid handling instrument (e.g., 1-channel, 8-channel, 96-channel, or 384-channel aspirator/dispenser). Such liquid handling produces flow velocity vectors parallel and opposite to the surface normal vector of the bottom surface of the well. In order to take full advantage of the protection from flow provided by the patterned structures, the velocity vector can be perpendicular to the surface normal vector. Such a flow pattern may be developed by providing a shelf-feature adjacent to the patterned structures (in this example an array of posts). As the flow front hits the shelf, the shelf deflects the flow front to the side such that the flow vectors in the patterned structure region become perpendicular to the surface normal vector of the bottom surface of the well. It is understood that the above description of the function of the shelf would apply to a variety of other embodiments.

These and still other advantages of the invention will be apparent from the detailed description and drawings. What follows is merely a description of preferred embodiments of the present invention. To assess the full scope of the invention, the claims should be looked to as the preferred embodiments are not intended to be the only embodiments within the scope of the claims.

### BRIEF DESCRIPTION OF THE DRAWINGS

FIG. 1 is four views of a microtiter plate including three enlarged views with one embodiment of patterned structures and shelf region.

FIG. 2 is multiple enlarged views of a number of alternative embodiments of the macrostructures (or microstructures) and shelves.

FIG. 3 is three alternative embodiments of formats wherein the macro- or micro-structures can reside.

FIG. 4 is a multiple view schematic of liquid handling to deposit suspended particles or cells into the retention area within a well.

FIG. 5 is a multiple view schematic of a liquid handling routine for changing the liquid in the well within which the particle or cell suspension resides.

FIG. 6 is a schematic of a 384-well microtiter plate format embodiment with a 384-tip liquid dispensing unit.

FIG. 7 is four views of a single well containing suspension cells at initial dispense (A, B) and after 10 washes with PBS (C, D). Two of the images have patterned macrostructures (A, C) while two do not (B, D).

FIG. 8 shows the retention percentage of non adherent cells following during immunocytochemistry after 13 wash steps (A). The image (B) shows immunocytochemical staining of non adherent cells following 13 wash steps. The image (C) shows the cell movement during immunocytochemistry after six washes.

### DETAILED DESCRIPTION

Various terms are used herein to refer to aspects of the present invention. To aid in the clarification of the description of the components of this invention, the following definitions are provided.

Flow velocity is used herein to describe the velocity of an element of fluid at a given position and time. The flow velocity u of a fluid is a vector field:

u=u(x,t)

which gives the velocity of an element of fluid at a 3-D position vector x and a time t.

Sheer force is used herein to describe a force vector that is perpendicular to the surface normal vector of the fluid element.

Micro-structures is used herein to describe features having at least one height, width, or length geometry in the range between 100 nanometers and 100 microns. Such features may include, but are not limited to, posts (cylindrical, elliptical, rectangular, polygonal cross-section), wells (cylindrical, elliptical, rectangular, polygonal cross-section), walls, baffles, honeycombs, domes, cones, and pyramids.

Macro-structures is used herein to describe features having at least one height, width or length geometry in the range between 0.10 mm and 10 mm. Such features may include, but are not limited to, posts (cylindrical, elliptical, rectangular, polygonal cross-section), wells (cylindrical, elliptical, rectangular, polygonal cross-section), walls, baffles, honeycombs, domes, cones, and pyramids.

Compartmentalization is used herein to describe a physical boundary between regions.

Patterned structures is used herein to indicate macro- or micro-structures arranged in a specific pattern, such as rectangular array (2-D), close-packed array (2-D), linear array (1-D), or randomized cluster with a specific range of spacing, such that the pattern has the desired fluid dynamic effects. The lowest surface of a well array is inside the wells. The lowest surface of a post array is in between the posts.

Wells is used to describe compartments within plates (such as, but not limited to 6-, 12-, 24-, 96-, 384- or 1536- format plates), slides, petri dishes or assay dishes which can contain fluid and have a physical boundary between nearby compartments. These wells may or may not include patterned structures.

The invention will now be described in detail with reference to a few preferred embodiments, as illustrated in the accompanying drawings. In describing the preferred embodiments, specific details are set forth in order to provide a thorough understanding of the invention. However, it will be apparent to one skilled in the art that the invention may be practiced without some or all of these specific details. In other instances, well-known features and/or process steps have not been described in detail so as not to unnecessarily obscure the invention. In addition, like or identical reference numerals are used to identify common or similar structural elements.

The devices and methods described herein exploit the fluid mechanical properties of patterned micro- or macro-structures to diminish movement and loss of particles or cells that have settled to the lowest surface of the patterned structures. While some or all of the particles or cells may in some cases adhere to the surface of the patterned structure, this device and method is particularly advantageous for particles or cells that remain non-adherent. In traditional assay devices, non-adherent cells are at risk of loss where no means are provided to protect the non-adherent cells from flow.

While liquid is being dispensed inside a well, a positive pressure is created between the orifice of the pipette tip and the distal areas of the well. This pressure is equilibrated by flow as the fluid moves out to the distal areas. The pressure difference between two arbitrary points in the patterned structure region (referred to as region R2 below) has the potential to produce flow along the lowest surface of the microstructures. This flow is diminished by increasing the resistance to flow of the patterned structures. The resistance to flow at the lowest surface increases with decreasing spacing of pattern elements, increasing the height of pattern elements, and increasing fluid viscosity; the flow velocity at the lowest surface decreases with decreasing dispensing flow rate. The device design and method parameters must take into account the desired particle and washing liquid properties as well as dispensing rate constraints for optimal performance. A shelf region (referred to as region R1 below) may be included to deflect the flow from the pipette before it reaches the patterned structure region. By including a shelf region, the flow velocity vectors will be perpendicular to the surface normal vector of the bottom surface of the well. Although not necessary, this condition is optimal for diminishing flow velocity at the lowest surface of the patterned structures where the particles or cells reside.

Biological cells are denser than cell culture media and thus settle out of suspension. However, their density varies, and with that the rate of settling. The drag on a particle or cell increases with increasing liquid density and increasing size. The device design and method parameters must take into account the properties of the particles or cells and the washing liquid for optimal performance.

In the instant invention, an assay vessel contains one or more assay compartments or wells (e.g., each well in a 96-well plate is an assay compartment), and each assay compartment has on its bottom surface one or more patterned structures, and optionally one or more shelves.

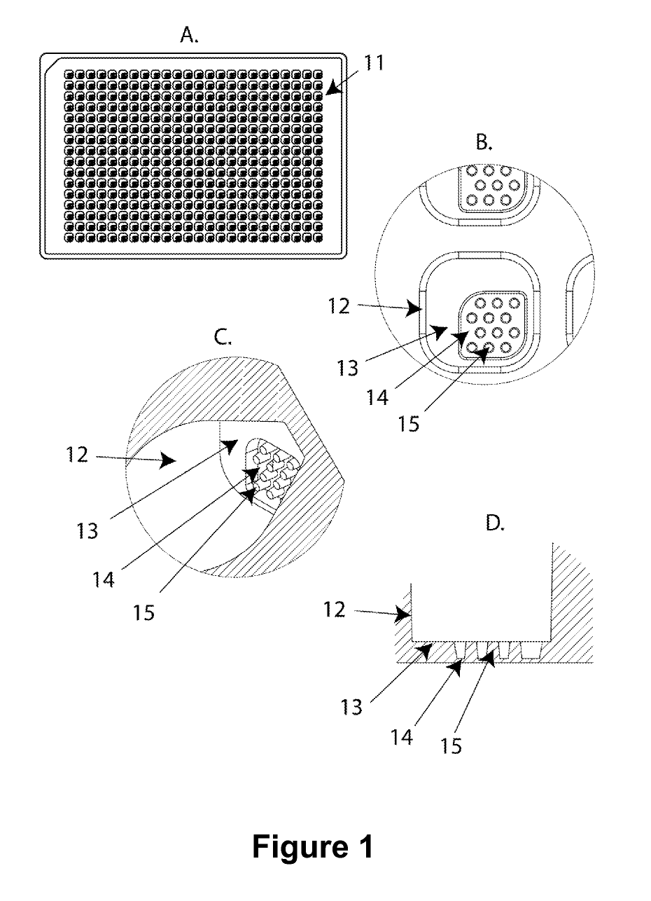

For the purposes of illustrating the principles of the invention, a representative embodiment of a 384-well microtiter plate format (11) with one possible design including a patterned structure is shown in FIG. 1. The bottom of the well (12) includes two regions: R1, which generally corresponds to a shelf (13), and R2, which generally corresponds to a recessed portion (14) of the well (12) having patterned structures such as posts (15). R2 is recessed relative to R1 and the tops of the posts (15) may or may not be level with the shelf (13). The region R1 may be a wide variety of different sizes and may also take various shapes and relationships relative to R2.

A representative embodiment may contain one or more shelves (13) at the bottom of the well (12), where the shelf or shelves (13) are elevated above the bottom surface of the recessed portion (14) of the well (12), where the shelf or shelves (13) are connected to the sidewall of the recessed portion (14) of the well (12), where the shelf or shelves (13) divide the recessed portion (14) of the well (12) into two or more segments, or where the shelf or shelves are not connected to the sidewall of the recessed portion (14) of the well (12).

In one embodiment, the ratio of the area divided between the shelf or shelves and the patterned structures is in the range of 1:100,000 to 100,000:1. In another embodiment, the ratio of the area divided between the shelf or shelves and the patterned structures is in the range of 1:100 to 100:1. In still another embodiment, the ratio of the area divided between the shelf or shelves and the patterned structures is in the range of 1:20 to 20:1.

It is understood that FIG. 1 is a structure shown for representative purposes only, and that the patterned structures may exist as a wide variety of number and shapes. Furthermore, R1 or the shelf (13) may in some cases be eliminated, and the benefits of the current invention gained from the patterned structures in R2 alone.

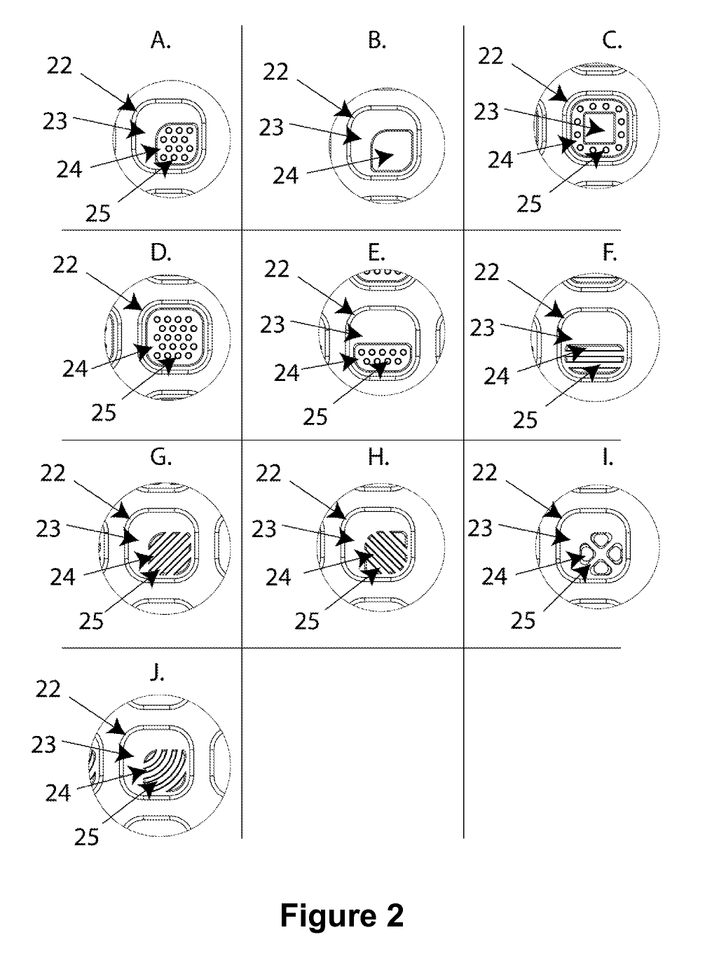

FIG. 2 shows ten selected embodiments of the regions R1 (23) and R2 (24) from FIG. 1 with various patterned structures (25). Patterned structures (25) can be posts as in FIGS. 2A, 2C, 2D, or 2E; linear baffles/walls as in FIGS. 2F, 2G, or 2H; intersecting walls as in FIG. 2I; or curved baffles/walls as in FIG. 2J.

Region R1, which includes the shelf, can also take various shapes and relationships relative to R2. Region R1 can isolate region R2 to a corner as in FIGS. 2A, 2B, 2G, 2H, 2I, and 2J; divide the well in half as in FIGS. 2E and 2F; be centrally located as in Figure C23; or alternatively the patterned structures in the region R2 can be sufficient to obviate the need for a separate region R1 as in FIG. 2D. Alternatively, region R2 may be shaped such that the need for additional patterned structures within the region R2 is unnecessary as in FIG. 2B. It is understood that these geometries are representational only, and that the design of R1 and R2 may have a wide variety of different geometries.

In one embodiment, the macro and micro structures within the recessed portion(s) of the well occur as arrays in the forms of linearly or offset arranged posts, linear baffles or walls, intersecting walls, curved baffles or walls, or with microwells or holes.

In one embodiment, the macro or micro structures within the recessed portion(s) of the well are comprised of an aspect ratio of height to width within the range of 1:1000 to 1000:1. In another embodiment, the macro or micro structures within the recessed portion(s) of the well are comprised of an aspect ratio of height to width within the range of 1:100 to 100:1. In still another embodiment, the macro or micro structures within the recessed portion(s) of the well are comprised of an aspect ratio of height to width within the range of 1:10 to 10:1. In still yet another embodiment, the macro or micro structures within the recessed portion(s) of the well are comprised of an aspect ratio of height to width within the range of 1:3 to 3:1.

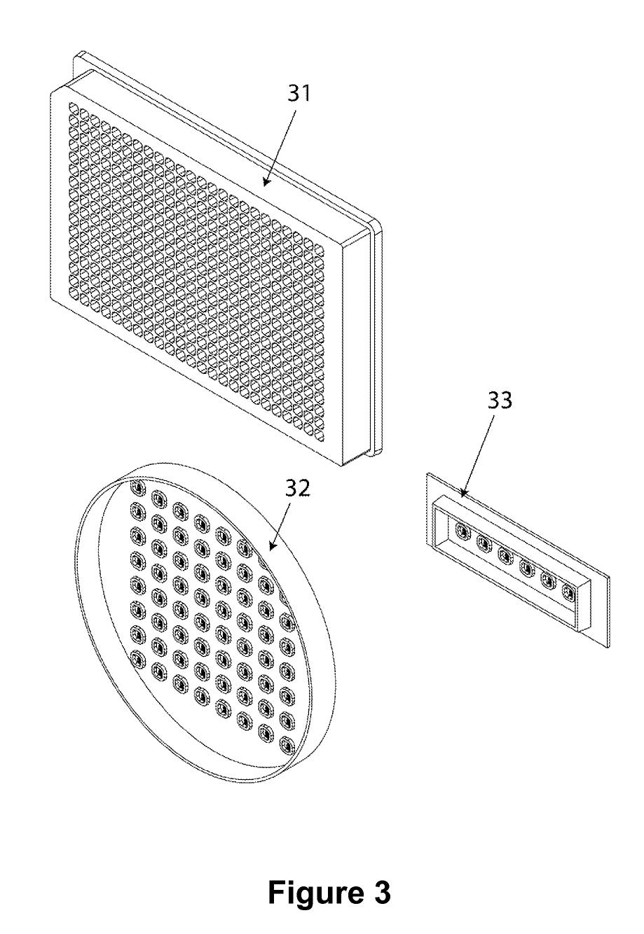

FIG. 3 shows a view of three selected formats which patterned macrostructures can reside upon. Cell biology experiments commonly utilize 384-well microtiter plate (31), petri dish (32) and slide (33) formats. It is understood that these formats are representational only, and that the format upon which the features are patterned may have a wide variety of different geometries. In one embodiment, the patterned microstructures are contained within a well. It is understood that the patterned macrostructures may also be addressed in bulk without individual compartmentalization of patterned macrostructures. It is understood that the patterned features may be macrostructures or microstructures. It is understood that compartmentalization is not limited to a well and may include, but is not limited to, other embodiments such as the use of a droplet of liquid restrained by a hydrophobic barrier.

In one embodiment, the well(s) range in size from those within a standard 3456-well plate (0.5-2 mm length/width/diameter by 3-4 mm height) to those within a standard bioassay dish (200-250 mm length/width by 20-30 mm height).

In another embodiment, the well(s) range in size from those within a standard 384-well plate (3-4 mm length/width/diameter by 11-12 mm height) to those within a standard petri dish (30-150 mm diameter by 12-20 mm height).

In another embodiment, the well(s) are square or round and are sized equivalent to those within a standard 384-well plate (3-4 mm length/width/diameter by 11-12 mm height).

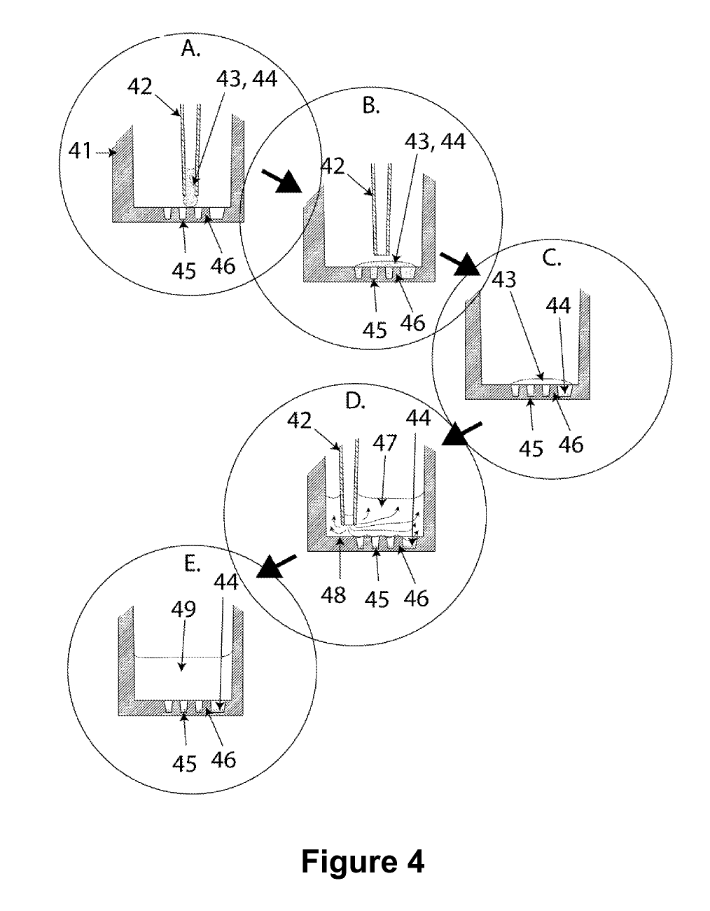

FIG. 4 is a schematic demonstrating liquid handling in a well (41) with patterned structures (46).

FIG. 4A shows a liquid dispensing pipette tip (42) carrying particles (44) in suspension in a liquid (43) when lowered within close proximity of region R2 (45) which contains patterned structures (46). It is understood that the dispensing tip (42) may have a wide variety of sizes and locations in 3-D space within close proximity of region R2 (45).

FIG. 4B shows that the dispensing tip (42) has dispensed the liquid (43) containing the particle in suspension (44) into region R2 (45) and surrounding the patterned structures (46). It is understood that the dispensing flow rate, volume, as well as R2 coverage, and well fill fraction may have a wide range of values.

FIG. 4C shows the particles (44), after some amount of time, have settled toward the bottom of region R2 (45) to the lowest surface of the patterned structures (46) leaving liquid without suspended particles (43) toward the top of region R2 (45).

FIG. 4D shows a liquid dispensing tip (42) dispensing liquid (47) in proximity of region R1 (48) adjacent to region R2 (45) which contains the particles (44) at the lowest surface of the patterned structures (46). Regions R1 and R2 are designed and the dispensing method is carried out in such a way that the liquid flow introduces minimal disturbance and maximum retention of the particles as the liquid flows over the patterned structures (46). It is understood that the dispensing tip may have a wide variety of sizes and locations in 3-D space within close proximity of region R1. It is understood that the dispensing flow rate, volume, as well as R2 coverage, and well fill fraction may have a wide range of values.

FIG. 4E shows the dispensed liquid (49) continuous with the liquid in which the particles were originally suspended. Diffusion can occur between the bulk liquid and region R2 (45) which contains the particles (44) at the lowest surface of the patterned structures (46).

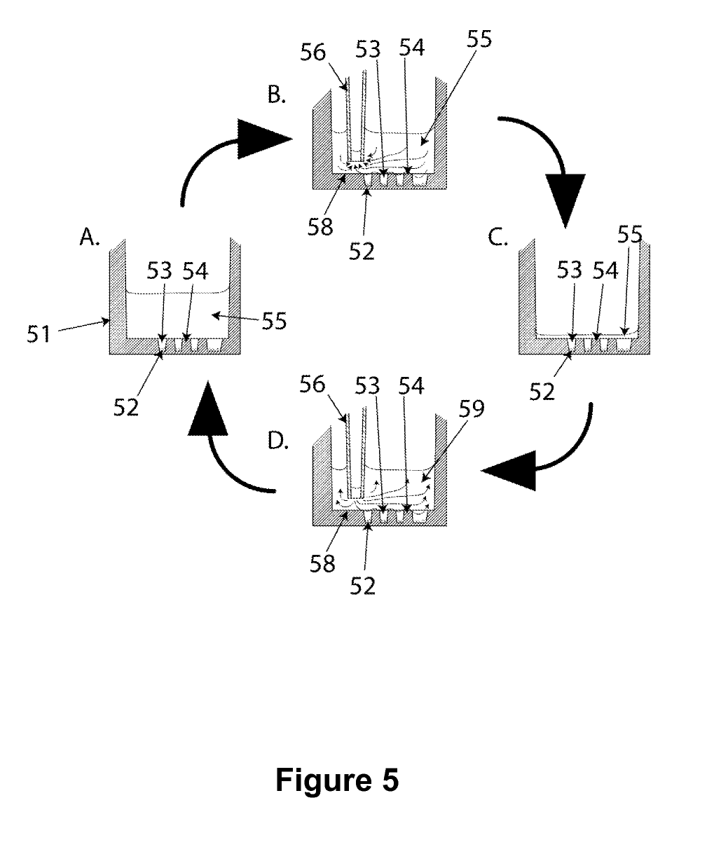

FIG. 5 is a schematic of liquid changes, or washing, in a well (51) with region R2 (53) containing particles (52) at the lowest level of the patterned structures (54).

FIG. 5A is a well (51) with region R2 (53) containing particles (52) at the lowest surface of the patterned structures (54) where the well is filled with a liquid (55). Such a state may be obtained by following the stepwise procedure illustrated in FIG. 4.

FIG. 5B shows a pipette tip (56) located in proximity to region R1 (58) withdrawing liquid (55) such that the flow introduces minimal disturbance and maximal retention of the particles (52) as it flows over the region R2 (53) which contains the patterned structures (54). It is understood that the pipette tip (56) may have a wide variety of sizes and locations in 3-D space within close proximity of R1. It is understood that the dispensing flow rate, volume, as well as R2 coverage, and well fill fraction may have a wide range of values.

FIG. 5C shows a low liquid level (55) with liquid still covering the particles (52) in region R2 (53) which contain the patterned structures (54).

FIG. 5D shows a pipette tip (56) located in proximity to region R1 (58) dispensing liquid (59) such that the flow introduces minimal disturbance and maximal retention of the particles (52) as it flows over the region R2 (53) which contains the patterned structures (54). It is understood that the dispensing tip may have a wide variety of locations in 3-D space. It is understood that the dispensing flow rate, volume, as well as R2 coverage, and well fill fraction may have a wide range of values.

It should be appreciated that multiple cycles of removal and introduction of fluids illustrated in FIG. 5 may be performed to, for example, introduce reagents into contact with the cells and/or particles, and subsequently remove the reagent and wash the cells and/or particles. Again, the introduction and removal of liquids in the well may be performed with little disturbance to the cells and/or particles residing at the bottom of the patterned structure. Once a liquid is presented in the main volume of the well, this liquid can diffuse into the recessed patterned region in a non-turbulent manner that does not disturb the cells and/or particles.

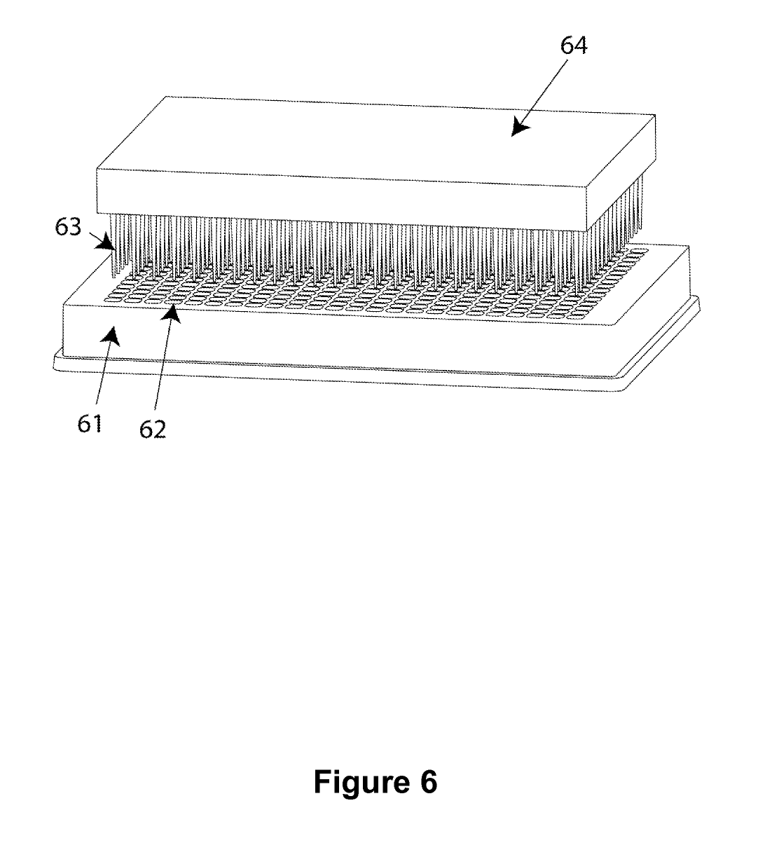

FIG. 6 shows a 384-well microtiter plate format (61) containing wells with patterned structures (62) with a 384-tip liquid dispensing unit (63) attached to a robotic liquid handler (64). It is understood that the liquid handler can be robotic or manual with a wide variety of tip number and configurations possible.

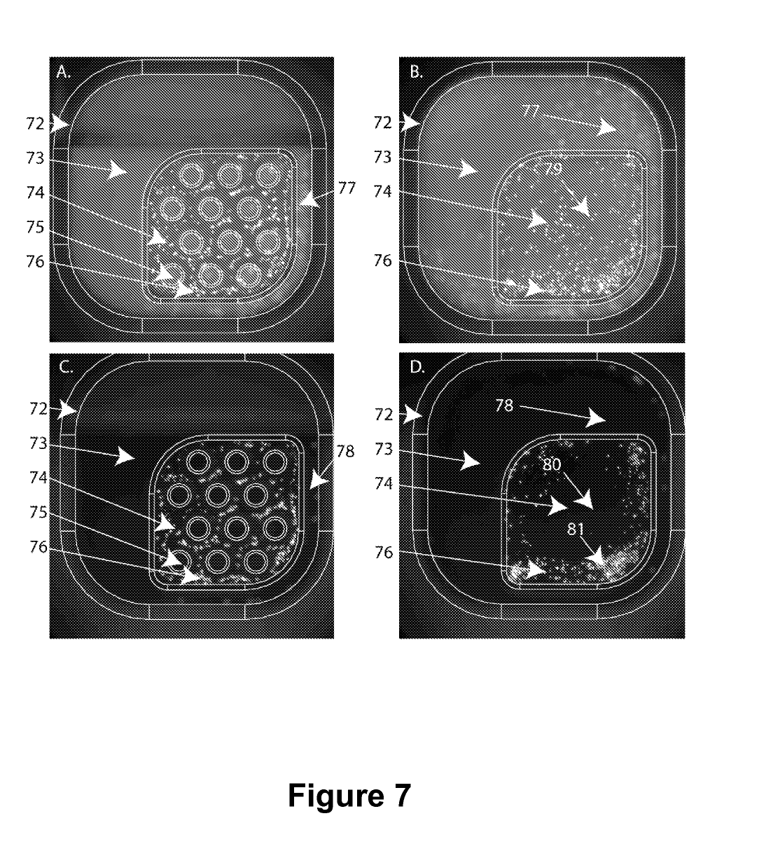

FIG. 7 shows four views of a single well from a 384-well plate which contains regions R1 (73) & R2 (74). Referring now to FIGS. 7A and 7B, a white line schematic of the well wall (72), region R1 (73) and region R2 (74), which contains patterned macrostructures (75) in FIG. 7A, is overlaid on photomicrographs of the actual well with cells (76) dispensed to it. A series of ten phosphate buffer saline (PBS) washes was executed using a 96-tip liquid dispensing robot. After the ten washes, the wells of FIGS. 7A and 7B were imaged again in FIGS. 7C and 7D, respectively. Ten washes was chosen as it was representative of a standard immuno-cytochemistry protocol. It is understood that the exact washing protocol used may have a wide range of liquid dispensing and removal steps, and a variety of reagents in various sequences. Actual dimensions of one embodiment are described later in the text.

FIG. 7A shows a well containing cells (76) dispensed to region R2 (74) with some cells (77) located in region R1 (73). Region R2 (74) contains patterned macrostructure posts (75). The background fluorescence is from Cell Tracker Green in the suspension solution from cell efflux.

FIG. 7B shows a well containing cells dispensed to region R2 (74) with some cells (79) located near the center of region R2 (74), some cells (76) near the edge of region R2 (74), and some cells (77) in region R1 (73). In this example, region R2 (74) does not contain patterned macrostructures. The background fluorescence is from Cell Tracker Green in the suspension solution from cell efflux.

FIG. 7C shows the same well as FIG. 7A following a series of ten washing steps. Few cells (78) remain in region R1 (73), while the cells (76) within region R2 (74) amongst the patterned macrostructure posts (75) remain largely in place, showing negligible loss or movement. The background fluorescence is no longer detectable, indicating that the dissolved Cell Tracker Green fluorescent substance has been removed which is indicative of successful washing. Actual retention data from one of the embodiments is described later in the text.

FIG. 7D shows the same well as FIG. 7B following a series of ten washing steps. Few cells (78) remain in region R1 (73). The center (80) of region R2 (74) no longer has cells present as they have moved or been removed during the washing steps. The edge (81) of region R2 (74) shows retention of cells. The background fluorescence is no longer detectable indicating that the dissolved Cell Tracker Green fluorescent substance has been removed, indicative of successful washing.

In a representative experiment, twelve wells (3.3 mm×3.3 mm×11.5 mm, L×W×H) containing a region R2 (2 mm×2 mm), and a region R1 (1.3 mm wide shelf along two walls of the well) in a 384-well plate had a series of liquid handling steps performed within them. In the layout presented in FIGS. 7A and 7B the wells contained a region R2 within which was a patterned macrostructure consisting of 12 tapered posts of the following design: 0.32 mm diameter at the base, 0.25 mm diameter at the top, 5 degree draft angle, 0.4 mm in height, 0.2 mm minimum distance between the base of posts, 0.18 mm minimum distance to the edge of region R2. A 1.6 μL, volume of cell suspension, pre-labeled with Cell Tracker Green, containing on average 985 cells (as counted by Count Nuclei using Metamorph software) was dispensed to region R2 as described in FIG. 4. During the dispensing, the tip was brought to about 0.100 mm above the top of the macro-structure posts. A series of ten washing steps using phosphate buffered saline in which 24 μL, was aspirated at 0.75 μL/sec flow rate and 20 μL, was dispensed at 0.75 μL/sec was executed above region R1 as shown in FIG. 5. During the dispensing, the tip was brought to about 0.200 mm above the top of the macrostructure posts. After the ten washes, another count was performed. On average, fifteen cells were lost with a standard deviation of eighteen cells. This is an average loss of 1.3% across the entire ten wash protocol. After two washes, the background fluorescence of Cell Tracker green was reduced by forty-nine fold, or approximately a seven-fold reduction per wash cycle. After two washes, the fluorescence was below the detection range of our camera and microscope. Under parallel conditions, analogous wells without the patterned structures lost the vast majority of cells as a result the same liquid dispensing routine.

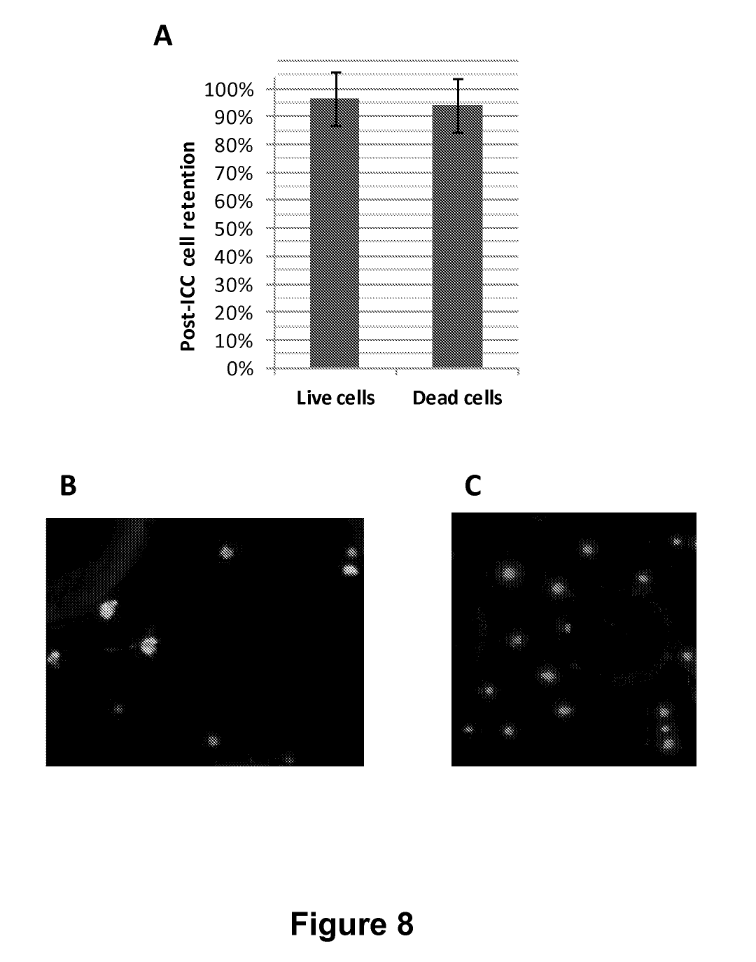

FIG. 8 shows the retention of cells following immunocytochemistry. A) Retention of cells following immunocytochemistry. Acute myeloid leukemia cells (PLB-985), a non adherent cell line, were loaded into the CellWasher plate after labeling with CellTracker Green (live cells); treatment with ethanol followed by DAPI following (dead cells) and imaged. Live and dead cells were subject to a full immunocytochemistry protocol including 13 wash steps and imaged again. Retention percentage is equal to the number of cells at the end of the immunocytochemistry protocol divided by the number prior to it. B) Immunocytochemical staining of PLB-985 cells. A standard immunocytochemistry protocol for β-actin, with 13 wash steps was used. Shown is an overlay of β-actin (green) and DAPI (blue). C) Cell movement during immunocytochemistry. PLB-985 cells were labeled with CellTracker Green, loaded into the CellWasher plate and imaged before and after six washes with PBS. Shown is an overlay of initial (blue) and final (red) cell positions.

In one embodiment, the instant invention may be used to wash cells or particles by: providing a volume of liquid to the well(s) containing particles or cells; providing an additional liquid volume; allowing the particles or cells to settle to the lowest surface of the macro or micro structures by gravity, centrifugation, affinity or magnetism; removing excess fluid volume; and replacing this fluid volume once or multiple times.

In one embodiment, the rate of dispensing and removing fluid volume is in the range of 10 nl/sec to 1 ml/sec. In another embodiment, the rate of dispensing and removing fluid volume is in the range of 50 nl/sec to 100 μl/sec. In another embodiment, the rate of dispensing and removing fluid volume is in the range of 100 nl/sec to 10 μl/sec.

In one embodiment, the loss of cells or particles following each wash cycle is less than 10% of the cells or particles present.

In one embodiment, the loss of cells or particles following five wash cycles is less than 10% of the cells or particle present.

In one embodiment, the loss of cells or particles following 10 wash cycles is less than 10% of the cells or particle present.

In one embodiment, the loss of cells or particles is less than 10% of the cells or particle present following 11, 12, 13, 14, 15, 20, 25, 30, 35, 40, 45 or even 50 wash cycles.

In one embodiment, the concentration of a dissolved substance present after a wash cycle is less than 50% of the concentration of that same substance before the wash cycle.

The device and method described here may be utilized to carry out immuno-cytochemical staining of non-adherent cell lines (e.g., HL-60). The cells would be cultured in the usual manner, suspended in cell culture media and dispensed to the device. The cells would be allowed to settle to the lowest surface the patterned structures. A series of wash steps would be executed using an automated (or manual) dispenser/aspirator. First, the cells would be fixed (e.g. using formalin), then permeabilized (e.g., using detergent such as Triton X-100), next the cells would be incubated with an appropriate blocking buffer (e.g. serum), followed by the primary antibody which recognizes the antigen of interest, and then the secondary antibody which carries the fluorescent dye (or enzyme). The last three steps are each followed by one or more washes with buffer (e.g., phosphate-buffered saline). In this example, the device and method facilitate the automation of the cell staining by eliminating the need to spin the cells down in a centrifuge after each wash step.

Another embodiment, of this application may be utilized to carry out assays with adherent cells without the loss of cells rendered non-adherent. This is important in many cell biology experiments, such as live/dead assays where quantification of programmed cell death is desired as it is well known that adherent cells become non-adherent when they undergo programmed cell death, or apoptosis. Here, the adherent cells would be seeded and grown on the lowest surface of the patterned structures using traditional cell culture methods. At the end of the assay, the cells could be stained with histochemical or immune-cytochemical reagents without loss of dead cells that were no longer adherent.

Another embodiment of this application involves the processing of particles suspended in a gel. Here, the posts would provide a scaffold which would protect the particle-containing gel from shear forces applied by liquid washing. The particles could be cancer cells, and the gel could be extra-cellular matrix (ECM) of a particular composition. Protocols involving fragile (e.g., low density) ECM compositions would benefit particularly from this invention.

Yet another embodiment of this application would involve partitioning the region R2 of the surface into two sub-regions, one containing patterned microstructures and the other not. This would simultaneously enable some particles or gels to be exposed to shear force and others not.

Yet another embodiment of this application would involve partitioning the region R2 of the surface into two sub-regions, each containing patterned microstructures, and separated by a gap or wall. This would enable co-culture of two different cell types either in two dimensions on the lowest surface of the patterned structures, or suspended in a gel. Region R2 could be further divided into an arbitrary number of subregions.

Another embodiment of this application would involve two distinct shelf regions R1a and R1b located adjacent to region R2. Region R2 could further contain a number of regions R1 adjacent to it.

Another embodiment of this application would involve patterning region R2 with various different patterned marco or micro structures. For example, a portion of region R2 may contain patterned macrostructures and another portion may contain patterned microstructures.

Another embodiment of this application would involve patterned structures in region R1. This includes, but is not limited to, the various micro and macro structures discussed above and below in the context of region R2.

Another embodiment of this application would involve subdividing the bottom surface of the assay well such that different sub-areas may be at different heights relative to one another; in this embodiment, regions R1 and/or R2 could be each cover one or more sub-areas.

A further embodiment may include a sub-region within region R2 which is designed to enable retrieval of non-adherent cells once the vessel is tilted (e.g., 45°) by aspirating the liquid in that sub-region with pipette tip. The region R2 would be resistant to flow when liquid is dispensed and aspirated in region R1. Region R2 would be designed with one continuous lowest surface and have a sub-region within R2 shaped such that when the vessel (e.g., microtiter plate) is tilted, the cells would roll over or be otherwise transported to the sub-region within R2 which could accommodate a pipette tip for aspiration of the cells.

It is contemplated that a device of the kind described herein may be formed by injection molding.

One embodiment of the fabrication process of a device containing the described features would be injection molding of all but the well walls and subsequent joining of the molded piece to another piece containing the well and tray/dish walls.

One embodiment of the fabrication process of a device containing the described features would be injection/compression molding of all but the well walls and subsequent joining of the molded piece to another piece containing the well walls.

One embodiment of the fabrication process of a device containing the described features would be hot embossing molding of all but the well walls and subsequent joining of the molded piece to another piece containing the well walls.

One embodiment of the fabrication process of a device containing the described features would be machining the device out of a piece, or pieces of material(s).

One embodiment of the fabrication process of a device containing the described features would involve using stereolithography to build the part via 3D printing processes.

One embodiment of the fabrication process of a device containing the described features would involve casting the device using soft lithography techniques.

An embodiment of the device that requires joining of two pieces could employ thermal bonding, adhesive bonding, ultrasonic welding, laser welding, plasma activated surface joining, solvent bonding, amongst other joining processes involving entanglement, covalent bonding, electrostatic interactions and Van der Waals interactions. It is understood that this is not a complete list of joining processes and that other processes may be employed to join the components.

One embodiment of device fabrication would have it fabricated in a thermoplastic such as Polystyrene, Polycarbonate, Cyclo-Olefin Polymer, PETG, PET. It is understood that there are many thermoplastics that could be used and that these are just a few examples of possible materials.

One embodiment of the device fabrication would require surface treatment to change the surface energy of the material that the device has been fabricated from. Example processes to change the surface energy can include coating the surface with a surfactant, coating the surface with a biomolecule, using corona discharge and using a plasma treatment system. It is understood that there are many surface treatment methods and that only a few examples of possible methods has been listed.

One embodiment of the device fabrication would require patterning of surface treatment to provide a border in conjunction with or in place of a well. Such patterning could be done via stamping, masking and applying surface treatment and laser ablation patterning. It is understood that there are many ways to pattern surface treatment and that only a few examples of possible methods has been listed.

While the invention has been described with respect to a limited number of embodiments, those skilled in the art, having benefit of this disclosure, will appreciate that other embodiments can be devised which do not depart from the scope of the invention as disclosed herein. It is understood that the particular embodiments disclosed herein are illustrative only, and the invention embraces as such forms thereof within the scope of the following claims.

Also, recitation of ranges of values herein are merely intended to serve as a shorthand method of referring individually to each separate value falling with the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable laws. Moreover, any combination of the above-described elements in all possible variations thereof is encompassed by the invention unless otherwise indicated herein or otherwise clearly contradicted by context.

### RELATED PUBLICATIONS

1. U.S. Pat. No. 7,695,627
2. U.S. Pat. No. 5,019,512
3. U.S. Patent Pub. 2009/0298166
4. U.S. Patent Pub. 2007/0231851
5. U.S. Patent Pub. 2010/0273667
6. Yeom J, Agonafer D D, Han J-H, and Shannon M A, “Low Reynolds number flow across an array of cylindrical microposts in a microchannel and figure-of-merit analysis of micropost-filled microreactors”, Journal of Micromechanics and Microengineering 19:065025 (2009).
7. Flemming R G, Murphy C J, Abrams G A, Goodman S L, and Nealey P F, “Effects of synthetic micro- and nano-structured surfaces on cell behavior”, Biomaterials 20:573-588 (1999).
8. Folch A and Toner M, “Microengineering of Cellular Interactions”, Annual Review of Biomedical Engineering, 2:227-256 (2000)
9. Chen C S, Mrksich M, Huang S, Whitesides G M, and Ingber D E, “Geometric control of cell life and death” Science 276:1425 (1997).
10. Tan J L, Tien J, Pirone D M, Gray D S, Bhadriraju K, and Chen C S, “Cells lying on a bed of microneedles: An approach to isolate mechanical force”, PNAS 100:1484-1489 (2003).
11. Thery M, Racine V, Pepin A, Piel M, Chen Y, Sibarita J-B, Bornens M, “The extracellular matrix guides the orientation of the cell division axis”, Nature Cell Biology 7:947-953 (2005).

We claim:

1. A device with patterned structures disposed to selectively diminish convective fluid flow, the device comprising:
at least one well configured to receive particles or cells, the at least one well having a bottom with at least one portion containing patterned structures disposed to allow the particles or cells to settle out of liquid suspension onto the lowest surface within the well,
wherein the at least one well is disposed to enable fluid delivery and removal in a volume surrounding the patterned structures and the patterned structures are disposed to diminish fluid flow at the lowest surface of the patterned structures thereby protecting the particles or cells residing thereon from fluid flow forces when fluid is delivered or removed from the volume surrounding the patterned structures.

2. The device of claim 1 wherein the patterned structures are either protrusions or depressions.

3. The device of claim 1 further comprising at least one shelf at the bottom of the well, where the at least one shelf is elevated above the bottom portion of the well, where the at least one shelf is connected to a sidewall of the well, where the at least one shelf divides the well into two or more segments, or where the at least one shelf is not connected to the sidewall of the well.

4. The device of claim 3 wherein the ratio of the area divided between the at least one shelf and the patterned structures is in a range of 1:100,000 to 100,000:1.

5. The device of claim 3 wherein the ratio of the area divided between the at least one shelf and the patterned structures is in a range of 1:100 to 100:1.

6. The device of claim 3 wherein the ratio of the area divided between the at least one shelf and the patterned structures is in a range of 1:20 to 20:1.

7. The device of claim 1 wherein the at least one well ranges in size from those within a standard 3456-well plate (0.5-2 mm length/width/diameter by 3-4 mm height) to those within a standard bioassay dish (200-250 mm length/width by 20-30 mm height).

8. The device of claim 1 wherein the at least one well ranges in size from those within a standard 384-well plate (3-4 mm length/width/diameter by 11-12 mm height) to those within a standard petri dish (30-150 mm diameter by 12-20 mm height).

9. The device of claim 1 wherein the at least one well are square or round and are sized equivalent to those within a standard 384-well plate (3-4 mm length/width/diameter by 11-12 mm height).

10. The device of claim 1 further comprising macro and micro structures within the at least one well, wherein the macro and micro structures occur as arrays in the forms of linearly or offset arranged posts, linear baffles or walls, intersecting walls, curved baffles or walls, or with microwells or holes.

11. The device of claim 1 wherein the macro or micro structures within the at least one well have an aspect ratio of height to width within the range of 1:1000 to 1000:1.

12. The device of claim 1 wherein the macro or micro structures within the at least one well have an aspect ratio of height to width within the range of 1:100 to 100:1.

13. The device of claim 1 wherein the macro or micro structures within the at least one well have an aspect ratio of height to width within the range of 1:10 to 10:1.

14. The device of claim 1 wherein the macro or micro structures within the at least one well have an aspect ratio of height to width within the range of 1:3 to 3:1.

15. A method for using the device of claim 1 comprising in any order, combination or number of permutations:
providing a volume of liquid containing particles or cells to the at least one well;
providing an additional volume of liquid;
allowing the particles or cells to settle to the lowest surface of the at least one well by gravity, centrifugation, affinity or magnetism; and
performing a wash cycle at least once, said wash cycle comprising removing an excess fluid volume and replacing at least a part of this removed fluid volume.

16. The method of claim 15 wherein the rate of dispensing and removing fluid volume is in the range of 10 nl/sec to 1 ml/sec.

17. The method of claim 15 wherein the rate of dispensing and removing fluid volume is in the range of 50 nl/sec to 100 μl/sec.

18. The method of claim 15 wherein the rate of dispensing and removing fluid volume is in the range of 100 nl/sec to 10 μl/sec.

19. The method of claim 15 wherein a loss of cells or particles following the wash cycle is less than 10% of the cells or particles present.

20. The method of claim 15 wherein a loss of cells or particles following 10 wash cycles is less than 10% of the cells or particles present.

21. The method of claim 15 wherein a loss of cells or particles following 15 wash cycles is less than 10% of the cells or particles present.

22. The method of claim 15 wherein the concentration of a dissolved substance present after the wash cycle is less than 50% of the concentration of the dissolved substance before the wash cycle.

23. A device for selectively diminishing convective fluid flow, the device comprising:
at least one well configured to receive particles or cells, the at least one well having a bottom with at least one portion disposed to allow the particles or cells to settle out of liquid suspension onto a lowest surface of the well,
wherein the at least one well is disposed to enable fluid delivery and removal in a volume surrounding the bottom portion of the well and the bottom portion is disposed to diminish fluid flow at the lowest surface thereof, thereby protecting the particles or cells residing thereon from fluid flow forces when fluid is delivered or removed from the volume surrounding the bottom of the well.
